(19) United States
(12) Patent Application Publication
(10) Pub. No.: US 2026/0207943 A1
(43) Pub. Date: Jul. 23, 2026

(54) SYSTEMS AND METHODS FOR IMPLANTING A LEAD FOR TREATING BLADDER AND/OR BOWEL DYSFUNCTION

(71) Applicant: INSPIRE MEDICAL SYSTEMS, INC., Golden Valley, MN (US)

(72) Inventors: Stephen Lorne Bolea, Excelsior, MN (US); Christopher Poletto, North Oaks, MN (US); Lee Erik Swanson, Minneapolis, MN (US)

(73) Assignee: INSPIRE MEDICAL SYSTEMS, INC., Golden Valley, MN (US)

(21) Appl. No.: 19/144,246

(22) PCT Filed: Dec. 29, 2023
(86) PCT No.: PCT/US2023/086480

§ 371 Date: Jun. 27, 2025

Related U.S. Application Data
(60) Provisional application No. 63/436,500, filed on Dec. 31, 2022.

(51) Int. Cl. A61N 1/36; A61N 1/05

(52) U.S. Cl. CPC A61N 1/3606; A61N 1/0556; A61N 1/36135

(57) ABSTRACT

Systems and methods for treating bladder and/or bowel dysfunction of a patient includes implanting a lead carrying one or more stimulation elements to apply stimulation energy to one or more target sites. In some examples, the lead can be implanted using one or more of a guidewire, a tear-away sheath, an open surgical approach, a laparoscopic procedure, or endoluminally.

A portion of the population suffers from bladder and/or bowel dysfunction, such as one or both of urinary incontinence (or bladder incontinence) and fecal incontinence (or bowel incontinence). Diet, training, slings, and drug therapies may fail to treat incontinence.

### BRIEF DESCRIPTION OF DRAWINGS

FIG. 1 is a schematic illustration of anatomy of a human pelvic region.

FIG. 2 is a schematic illustration of the pelvic region of FIG. 1 and various nerves.

FIG. 3 is a block diagram of a treatment system in accordance with principles of the present disclosure.

FIGS. 4A-4F illustrate delivery systems and methods in accordance with principles of the present disclosure for delivering a lead to anatomy of a patient.

FIG. 5 is a simplified side view of a delivery system in accordance with principles of the present disclosure for delivering a lead.

FIG. 6 illustrates an open surgical method in accordance with principles of the present disclosure for delivering a lead to anatomy of a patient.

FIG. 7 illustrates open surgical methods in accordance with principles of the present disclosure for delivering a lead to anatomy of a patient.

FIGS. 8A-8C illustrate laparoscopic systems and methods in accordance with principles of the present disclosure for delivering a lead to anatomy of a patient.

FIGS. 9A-9B illustrate laparoscopic systems and methods in accordance with principles of the present disclosure for delivering a neurostimulator to anatomy of a patient.

FIGS. 10-10A illustrate endoluminal methods in accordance with principles of the present disclosure for delivering a lead to anatomy of a patient.

FIG. 11 is a simplified side view of a ribbon-mesh type lead.

FIG. 12A is a simplified end view of the lead of FIG. 11 loaded to an introducer tool in accordance principles of the present disclosure.

FIG. 12B is a simplified side view of a portion of the arrangement of FIG. 12A.

FIG. 12C is a simplified top view of a portion of the arrangement of FIG. 12A.

FIG. 13 diagrammatically illustrates a closed loop implant assist system and method in accordance with principles of the present disclosure.

FIG. 14 is a flow diagram schematically representing a closed loop implant assist method in accordance with principles of the present disclosure.

FIG. 15 diagrammatically illustrates a closed loop implant assist system and method in accordance with principles of the present disclosure.

FIG. 16 diagrammatically illustrates a closed loop implant assist system and method in accordance with principles of the present disclosure.

FIG. 17 is a block diagram schematically representing a care engine of a control portion.

FIG. 18 is a diagram schematically representing a patient's body, implantable components, and/or external elements of an example device and/or for use in an example method

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to implantable devices for diagnosis, therapy, and/or other care of medical conditions. At least some examples may comprise implantable devices and/or methods of implanting devices useful for treating bladder or bowel dysfunctions, including one or both of urinary incontinence and fecal incontinence of a patient, or other pelvic disorders. At least some such examples comprise implanting an electrode to deliver a nerve-stimulation signal to one or more nerves or nerve branches to activate a corresponding external sphincter, such as a branch of the pudendal nerve that activates the external urethral sphincter and/or the external anal sphincter. In some embodiments, operation of the implantable device is controlled in response to sensed information of the patient.

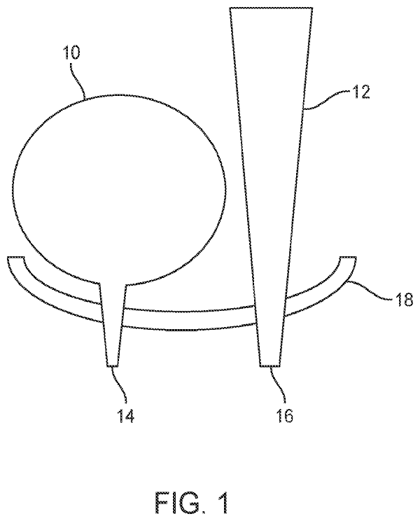

With reference to the greatly simplified view of FIG. 1, the human pelvic region includes a bladder 10 and a rectum 12. Contents of the bladder 10 are evacuated through a urethra 14, whereas contents of the rectum 12 are evacuated through the anus 16. Pelvic floor muscles 18 support the pelvic organs and span the bottom of the pelvis. The pelvic floor muscle layer 18 has holes for passage of the urethra 14 and the anus 16, and normally wraps quite firmly around these holes to help keep the passages shut.

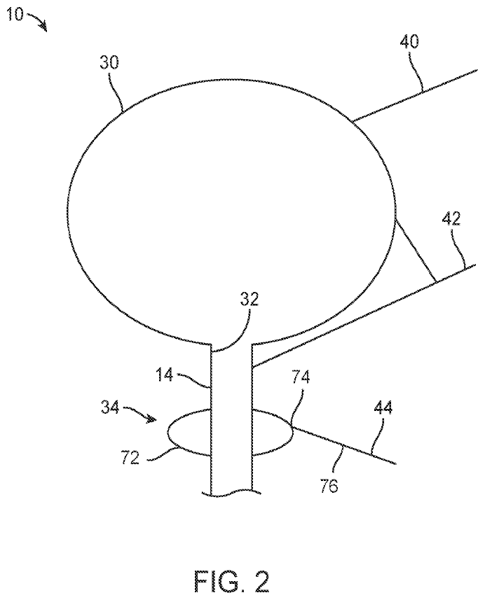

With additional references to the greatly simplified view of FIG. 2, the bladder 10 is a hollow muscular organ connected to the kidneys by the ureters. The detrusor 30 muscle (referenced generally) is smooth muscle found in the wall of the bladder 10. The urethra 14 is a tube or duct by which urine is conveyed out of the body from the bladder 10. Internal and external sphincters control flow of urine through the urethra 14; under normal conditions, when either of these muscles contract, the urethra 14 is sealed shut. In particular, an internal urethral sphincter (IUS) 32 (referenced generally) is a smooth muscle that constricts the internal orifice of the urethra 14. The IUS 32 is located at the junction of the urethra 14 with the bladder 10 and is continuous with the detrusor muscle 30, but is anatomically and functionally fully independent from the detrusor muscle 30. An external urethral sphincter (EUS) 34 is located in the deep perineal pouch, at the bladder's 10 distal inferior end around the mid urethra in females and inferior to the prostate in males. Urine is excreted from the kidneys and stored in the bladder 10 before elimination via the urethra 14 during what is known as the micturition reflex. During periods of bladder filling, the storage of urine is promoted by the actions of the internal and external urethral sphincters 32, 34 and the pelvic floor musculature 18. During micturition, these sphincters 32, 34 relax and the smooth muscle of the bladder (the detrusor muscle 30) contracts, resulting in the expulsion of urine.

The body of the bladder 10 is directly innervated by efferent fibers that arise from parasympathetic postganglionic neurons in the pelvic ganglia and intramural ganglia and by efferent fibers that arise from sympathetic postganglionic neurons in the lumbosacral sympathetic chain and hypogastric ganglia/pelvic ganglia. This is generally reflected in FIG. 2 by reference to a pelvic nerve 40 and a hypogastric nerve 42. The internal urethral sphincter 32 receives innervation from the hypogastric nerve 42. The external urethral sphincter 34 is directly innervated by motor neurons in the sacral segments of the spinal cord via the pudendal nerve 44.

Urinary continence is generally defined as the act of storing urine in the bladder 10 until the bladder 10 can be appropriately evacuated. Urinary continence requires control of the detrusor muscle 30 and is the result of complex coordination between multiple centers in the brain, brain stem, spinal cord, and peripheral nerves. As described above, micturition is a coordinated act of bladder elimination that involves relaxing the pelvic floor muscles 18, contracting the detrusor muscle 30, and simultaneously opening the urethral sphincters 32, 34 to achieve complete emptying of the bladder. Stress incontinence can be defined as the involuntary leakage of urine from the bladder 10 accompanying physical activity (e.g., laughing, coughing, sneezing, etc.) which places increased pressure on the abdomen. The leakage occurs even though the bladder muscles (detrusor muscle 30) is not contracting and an urge to urinate is not present. Stress incontinence can develop when the urethral sphincters 32, 34, the pelvic floor muscles 18, or all of these structures have been weakened or damaged and cannot dependably hold in urine. With urethral hypermobility, the bladder 10 and urethra 14 shift downward when abdominal pressure rises, and there is no hammock-like support for the urethra 14 to be compressed against to keep it closed. With urethral incompetence, problems in the urinary sphincter 32, 34 keep it from closing fully or allow it to pop open under pressure. Urinary urge incontinence (“UUI”) (sometimes referred to as overactive bladder (“OAB”) or detrusor overactivity) entails the involuntary leakage of urine from the bladder 10 when a sudden strong need to urinate is felt. There is a sudden involuntary contraction of the muscular wall (the detrusor 30) of the bladder that signals an immediate need to urinate, which can happen even when the bladder 10 is not full. Mixed incontinence is the term used to a combination of both overactive bladder and stress incontinence.

Internal and external sphincters are similarly provided with the anus 16 (i.e., the internal anal sphincter and the external anal sphincter), acting to keep the anal canal and orifice closed. Action of the internal anal sphincter (IAS) is entirely involuntary, and it is in a state of continuous maximal contraction. The external anal sphincter (EAS) is always in a state of contraction, but can be voluntarily put into a condition of greater contraction so as to more firmly occlude the anal orifice. Similar to urinary continence, bowel continence is the act of storing feces until an acceptable time and opportunity for elimination. Bowel continence requires competent internal and external sphincters, pelvic floor musculature, and intact neurological pathways. Neurological control of bowel continence is complex and requires coordinated reflex activities from the autonomic and enteric nervous systems. The colon can be visualized as a closed, pliant tube bounded by the ileocecal valve and the anal sphincter. The continuous, smooth muscle layer at the end of the rectum 12 thickens to form the internal anal sphincter (IAS); the external anal sphincter (EAS) is a circular band of striated muscle that contracts with the pelvic floor. Parasympathetic stimulation of the IAS from the pelvic plexus originates from the sacral cord (S1 to S2). Sympathetic stimulation of the IAS causes contraction. The EAS is composed of both smooth and striated muscle. The smooth muscle of the EAS is innervated by the enteric nervous system. The striated component of the EAS is innervated by the pudendal nerve that exits the cord at sacral levels S2, S3, and S4.

Fecal incontinence can be defined as the involuntary loss of rectal contents (feces, gas) through the anal canal and the inability to postpone an evacuation until socially convenient. For example, injuries to one or both of the EAS and IAS may make it difficult to hold stool back properly. Injury to the nerves that sense stool in the rectum or those that control the anal sphincter can also lead to fecal incontinence. A generalized weakness of the pelvic floor 18 can lead to an impaired barrier to stool in the rectum 12 entering the anal canal, and this is associated with incontinence to solids. The pelvic floor 18 is innervated by the pudendal nerve and the S3 and S4 branches of the pelvic plexus. If the pelvic floor muscles 18 lose their innervation, they cease to contract and their muscle fibers are in time replaced by fibrous tissue, which is associated with pelvic floor weakness and incontinence.

With the above in mind, various treatment systems and methods have been disclosed that treat bladder and/or bowel dysfunction (e.g., one or more of urinary incontinence, UUI and fecal incontinence) by supplying stimulation signals to an electrode implanted to apply the stimulation signal to one or more nerves and/or muscles of the patient that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). Examples of such systems and methods are provided in PCT Publication No. 2020/243104 (Rondoni, et al.) and PCT Publication No. WO 2022/192726 (Rondoni, et al.) the entire teachings of each of which are incorporated herein by reference.

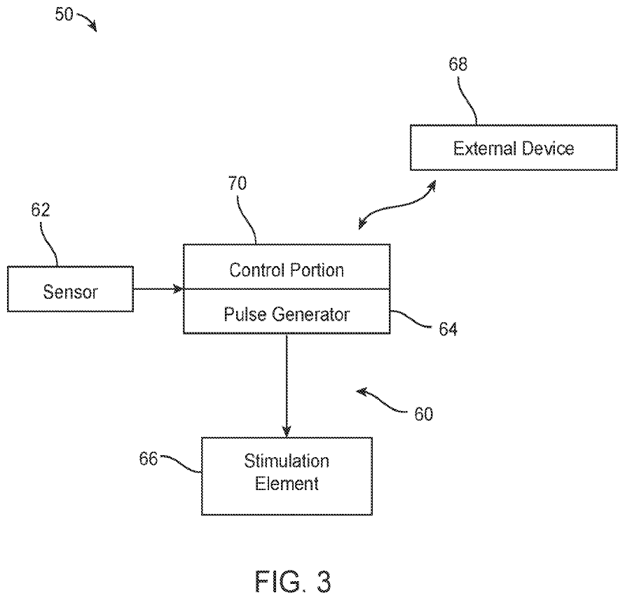

One example of a treatment system 50 for treatment of bladder and/or bowel dysfunction in accordance with principles of the present disclosure is provided in FIG. 3 and includes an implantable medical device (IMD) 60 (referenced generally) and optionally one or more sensors 62 (e.g., one or more of an accelerometer, a pressure sensor, a strain sensor, bioimpedance sensor, etc.). In general terms, the IMD 60 includes an implantable pulse generator or implantable component of a pulse generator (collectively identified as “IPG”) 64 and one or more stimulation elements (e.g., electrode or electrode assembly) 66. The IPG 64 is configured for implantation into a patient, and is configured to provide and/or assist in the performance of therapy to the patient. With formats in which the IPG 64 is an implantable pulse generator, a power source (e.g., battery) is carried within a housing of the implantable pulse generator and from which stimulation energy is generated. With formats in which the IPG 64 is an implantable component of a pulse generator, the implantable component(s) can include a receiver unit (e.g., receiver coil or similar device) that receives a signal from an external device (external the patient) that typically would be positioned on top of the skin over the location of the receiver coil. The external device can generate/deliver the stimulation energy at desired setting (e.g., amplitude, pulse width, frequency, pulse train length, etc.) to be received by the implanted receiver unit and conducted to the stimulation element(s) 66 for activation of tissue. The implanted receiver unit may or may not operate to modify the signal it receives prior to delivery to the stimulation element(s) 66. The external transmitter/controller may receive sensing signals from external sensor, receive sensing signals from the implanted portion of the implantable component via telemetry, etc. Unless stated otherwise, reference to “IPG 64” is inclusive of both an implantable pulse generator and an implantable component of a pulse generator as described above. The stimulation element 66 is configured to be implanted proximate a selected segment or region of the patient's anatomy, and is electrically connected to the IPG 64, for example via a lead. In other embodiments, the IPG 64 and the stimulation element 66 can be provided as components of a single or integral device, such as a microstimulator, as are known in the art. The IPG 64 is programmed to deliver (or is prompted to deliver) stimulation signals to the stimulation element 66 that in turn apply the signal. In some embodiments, the IPG 64 is programmed (or is prompted) to initiate, cease and/or modulate (e.g., titrate) delivered stimulation signals based upon one or more physical parameters of the patient. In this regard, the sensor(s) 62 sense the physical parameter of interest, and signal the so-sensed parameter to the IPG 64 (or other component controlling operation of the IPG 64). The sensor(s) 62 can be carried by the IPG 64, can be connected to the IPG 64, or can be a standalone component not physically connected to the IPG 64. The sensor(s) 62 can be self-contained and can communicate with the IPG 64 in some optional embodiments. In some embodiments, the sensor(s) 62, the IPG 64, and the stimulation element 66 can be provided as components of a single or integral device. In some embodiments, the treatment system 50 can further include an optional external device 68. Where provided, the external device 68 can, in some non-limiting embodiments, wirelessly communicate with the IMD 60.

The IPG 64 can assume various forms known in the art for generating a nerve-stimulating signal for delivery to the stimulation element(s) 66. For example, the IPG 64 can include a sealed case or enclosure maintaining a power source (e.g., battery) and electrical/circuitry components appropriate for formatting energy from the power source as the desired stimulation signal (e.g., a nerve-stimulation signal). In some embodiments, the IPG 64 as provided as part of, or is electronically linked to, a control system that includes a control portion 70 providing one example implementation of a control portion forming a part of, implementing, and/or generally managing stimulation element(s), power/control elements (e.g., pulse generators, microstimulators), sensors, and related elements, devices, user interfaces, instructions, information, engines, elements, functions, actions, and/or methods, as described throughout examples of the present disclosure. In some examples, the control portion 70 includes a controller and a memory. In general terms, the controller comprises at least one processor and associated memories. The controller is electrically couplable to, and in communication with, memory to generate control signals to direct operation of at least some of the stimulation elements, power/control elements (e.g., pulse generators, microstimulators) sensors, and related elements, devices, user interfaces, instructions, information, engines, elements, functions, actions, and/or methods, as described throughout examples of the present disclosure. In some non-limiting examples, these generated control signals include, but are not limited to, employing instructions and/or information stored in the memory to at least direct and manage treatment of bladder and/or bowel dysfunction by stimulating nerve(s), nerve branch(es) and/or muscle(s), for example to activate one or more of the external urethral sphincter 34 and the external anal sphincter, and/or pelvic floor nerves (e.g., the pudendal nerve 44, the sacral nerve) to relax the detrusor muscle 30 and prevent or reduce urgency or frequency.

In some instances, the controller or control portion 70 may sometimes be referred to as being programmed to perform the actions, functions, routines, etc. of the present disclosure. In some examples, at least some of the stored instructions are implemented as, or may be referred to as, a care engine, a sensing engine, monitoring engine, and/or treatment engine. In some examples, at least some of the stored instructions and/or information may form at least part of, and/or, may be referred to as a care engine, sensing engine, monitoring engine, and/or treatment engine.

In response to or based upon commands received via a user interface and/or via machine readable instructions, the controller generates control signals as described above in accordance with at least some of the examples of the present disclosure. In some examples, the controller is embodied in a general purpose computing device while in some examples, the controller is incorporated into or associated with at least some of the stimulation elements, power/control elements (e.g. pulse generators, microstimulators), sensors, and related elements, devices, user interfaces, instructions, information, engines, functions, actions, and/or method, etc. as described throughout examples of the present disclosure.

For purposes of the present disclosure, in reference to the controller, the term “processor” shall mean a presently developed or future developed processor (or processing resources) that executes machine readable instructions contained in a memory. In some examples, execution of the machine readable instructions, such as those provided via the memory of the control portion 70 cause the processor to perform the above-identified actions, such as operating the controller to implement the sensing, monitoring, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by the memory. In some examples, the machine readable instructions may comprise a sequence of instructions, a processor-executable machine learning model, or the like. In some examples, the memory comprises a computer readable tangible medium providing non-volatile storage of the machine readable instructions executable by a process of the controller. In some examples, the computer readable tangible medium may sometimes be referred to as, and/or comprise at least a portion of, a computer program product. In other examples, hard wired circuitry may be used in place of or in combination with machine readable instructions to implement the functions described. For example, the controller may be embodied as part of at least one application-specific integrated circuit (ASIC), at least one field-programmable gate array (FPGA), and/or the like. In at least some examples, the controller is not limited to any specific combination of hardware circuitry and machine readable instructions, nor limited to any particular source for the machine readable instructions executed by the controller.

In some examples, the control portion 70 may be entirely implemented within or by a stand-alone device.

In some examples, the control portion 70 may be partially implemented in the IPG 64 and partially implemented in a computing resource separate from, and independent of, the IPG 64. For instance, in some examples the control portion 70 may be implemented via a server accessible via the cloud and/or other network pathways. In some examples, the control portion 70 may be distributed or apportioned among multiple devices or resources such as among a server, a neurostimulation or neuromodulation treatment device (or portion thereof), and/or a user interface.

In some examples, the control portion 70 is entirely implemented within or by the IPG 64 (thereby defining an IPG assembly), which has at least some of substantially the same features and attributes as a pulse generator (e.g., power/control element, microstimulator) as described throughout the present disclosure. In some examples, the control portion 70 is entirely implemented within or by a remote control (e.g., a programmer) external to the patient's body, such as a patient control and/or a physician control (e.g., the external device 68). In some examples, the control portion 70 is partially implemented in the IPG 64 assembly and partially implemented in the remote control (at least one of the patient control and the physician control).

The systems and methods of the present disclosure are in no way limited to a particular stimulation target site(s) or a particular stimulation therapy regimen. The stimulation therapies or algorithms programmed to, or implemented by, the control portion 70 can be of any format deemed useful for the patient being treated, and may or may not act upon information from the sensor(s) 62. With reference between FIGS. 1-3, the system 50 can be configured and implanted to provide stimulation therapy to one or more nerves and/or muscles that, for example, influence the behavior of musculature of the pelvic region of the patient, for example musculature relating to one or both of urinary incontinence and fecal incontinence (e.g., the external urethral sphincter 34, the internal urethral sphincter 32, pelvic floor muscles 18, the external anal sphincter, the internal anal sphincter, etc.). For example, stimulation can be provided to one or more of the pudendal nerve 44, the pelvic nerve 40, the sacral nerve, hypogastric, or branches thereof. For example, stimulation can be provided to a deep branch of the pudendal nerve 44 or other nerve, for example applied to a distal-most branch of the pudendal nerve 44 (or other nerve) at or in highly close proximity to a location where the branch contacts or terminates a muscle (or other anatomical feature) of interest. With optional embodiments in which the treatment system 50 is configured and implanted to deliver stimulation to two (or more) target sites (e.g., two or more of the pudendal nerve 44, the pelvic nerve 50, the sacral nerve, the hypogastric nerve, etc., and/or two or more different locations along one incontinence amelioration-related nerve and/or different incontinence amelioration-related nerves, etc.), the so-applied simulation can be toggled (e.g., simultaneous, alternating, overlapping, unilateral, bilateral, selective), optionally while additionally toggling/adjusting one or more stimulation parameters e.g., amplitude, frequency, pulse width, duty cycle, pulse shape, etc.). Alternatively or in addition, the system 50 can apply electrical stimulation to tissue sites proximate a nerve or nerve branch of interest. In yet other embodiments, stimulation can be applied directly to a muscle. Various, non-limiting examples of stimulation protocols or algorithms are described in PCT Publication No. 2020/243104 (Rondoni, et al.) and PCT Publication No. WO 2022/192726 (Rondoni, et al.) the entire teachings of each of which are incorporated herein by reference.

The stimulation element(s) 66 can assume various forms appropriate for applying electrical stimulation to the anatomical feature (e.g., nerve) of interest, and can be provided as part of, or carried by a lead or lead assembly or the like. The stimulation element(s) 66 can be or include one or more electrodes in the form of ring electrodes, segmented electrodes, partial ring electrodes, coil electrodes and helical electrodes. In some examples, the stimulation element(s) may be or include a cuff electrode, comprising at least some of substantially the same features and attributes as described in Bonde et al., U.S. Pat. No. 8,340,785, Self Expanding Electrode Cuff, issued on Dec. 25, 2012 and Bonde et al., U.S. Pat. No. 9,227,053, Self Expanding Electrode Cuff, issued on Jan. 5, 2016, both which are hereby incorporated by reference in their entirety. Moreover, in some examples a stimulation lead, which may comprise one example implementation of a stimulation element, may comprise at least some of substantially the same features and attributes as the stimulation lead described in U.S. Pat. No. 6,572,543 to Christopherson et al., and which is incorporated herein by reference in its entirety. Other non-limiting examples of stimulation elements and leads useful with the present disclosure are provided in PCT Publication No. 2020/243104 (Rondoni, et al.) and PCT Publication No. WO 2022/192726 (Rondoni, et al.) the entire teachings of each of which are incorporated herein by reference.

With the above generalities in mind, the lead can be delivered and implanted in various manners to position the stimulation element(s) 66 at an intended target site, for example along the pudendal nerve 44. Aspects of the present disclosure provide for systems and methods for delivering/implanting a lead as part of the bladder and/or bowel disorder treatment system 50, so as to locate the stimulation element 66 (as provided, for example, as part of a lead, a cuff electrode, a microstimulator, etc.) at an intended target site. In some embodiments, laparoscopic procedures and delivery tools can be employed for the delivery of, for example, any of the leads or cuff electrodes of the present disclosure. In yet other embodiments, the stimulation element(s) can be provided as part of a trialing system that need not necessarily include the sensor(s) 62. With any of the embodiments of the present disclosure, a body of the lead can be relatively flexible; as part of a delivery and/or implant procedure, a stiffening wire can be inserted into the lead body. The so-supported lead can then more readily be directed to a target site. Once placed at a desired location, the stiffening wire can be removed from the lead body, so that upon final implant, the lead is highly flexible and more likely to stay in place.

In some embodiments, delivery systems of the present disclosure can include various tools, such as a needle, a guide (e.g., akin to a guidewire), and an introducer that are useful for placement of a stimulation lead (e.g., a lead carrying stimulation element(s), such as electrodes). With these and related embodiments (for example for the delivery of any of the leads of the present disclosure), the needle tool can include a needle body attached to a handle, with the needle body coated with an electrically insulator material except at the tip. The needle tip is inserted into the patient and advanced to the expected stimulation site. Electrical energy is then delivered through the needle at the tip to stimulate tissue and evaluate whether or not the needle tip is at the desired location. Once it is confirmed that the needle tip is located at the desired target site, a guide is inserted into and through a lumen of the needle and located at the attained target site, and the needle removed. In other embodiments, the handle can be removed from the needle body, such that the needle body can serve as a guide. Regardless, an introducer is inserted over the guide and advanced to locate a distal end of the introducer at the attained target site. The guide is removed, and the lead introduced and advanced to the attained target site through the introducer. In some optional embodiments, the so-positioned lead can be operated to stimulate tissue, allowing the clinician to confirm desired location of the lead before deploying anchor(s) carried by the lead. These techniques can be useful with any of the stimulation target sites of the present disclosure.

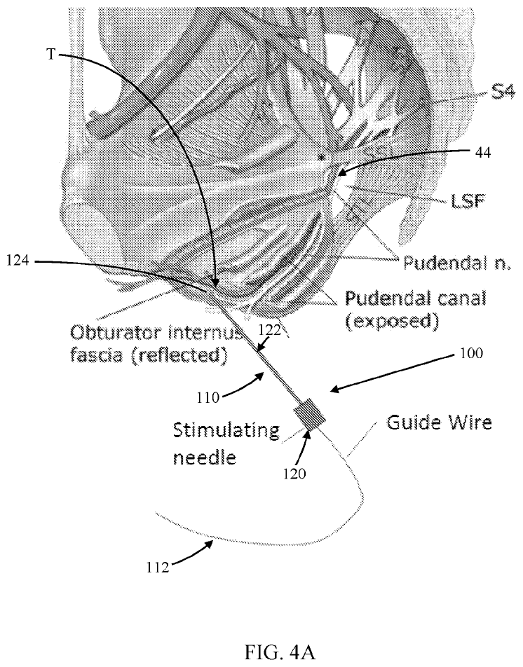

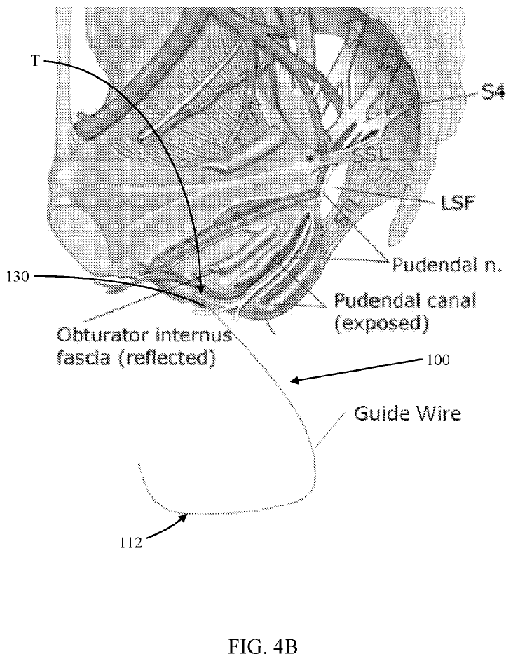

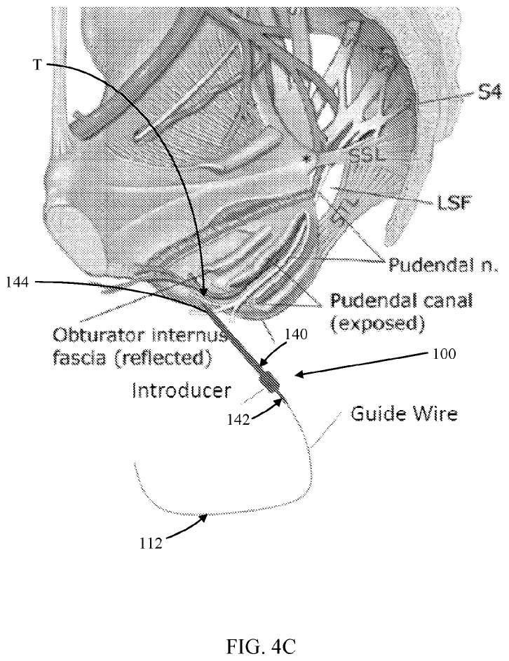

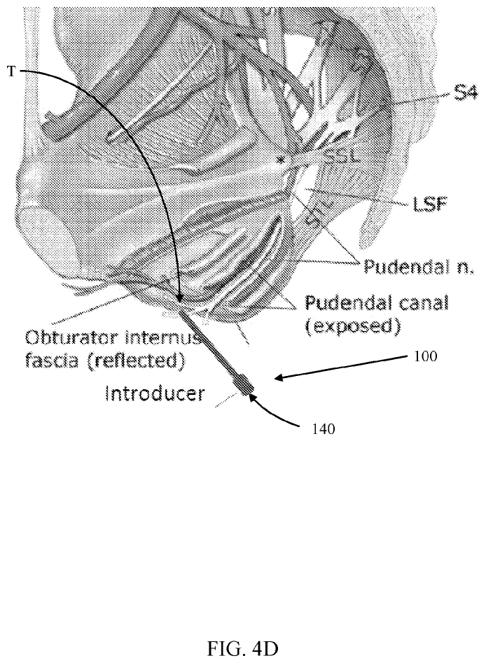

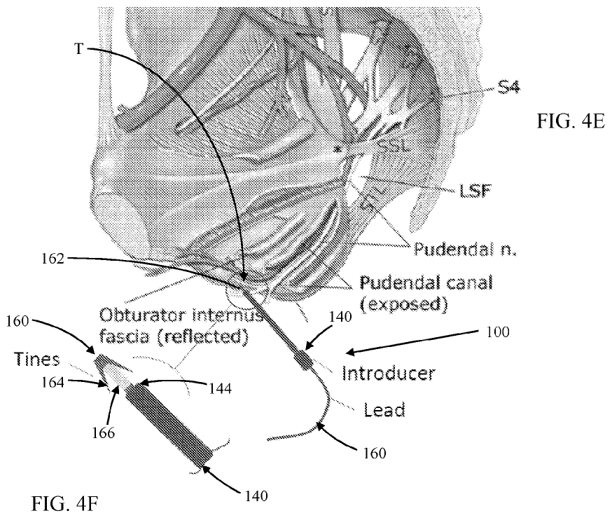

For example, FIG. 4A illustrates portions of one embodiment of a delivery system 100 for placing a lead (not shown) that, following implant, applies stimulation to an intended target site T, for example a stimulation lead as part of a trialing system or an implanted treatment system. In the non-limiting example of FIG. 4A, the intended target site T is a deep perineal branch of the pudendal nerve 44. The delivery system 100 includes a stimulation needle 110 and a guidewire 112. The stimulation needle 110 can be of a conventional design, and includes a handle 120 and a needle body 122 terminating at a tip 124. A lumen or bore (hidden) extends through the stimulation needle 110 and is sized to slidably receive the guidewire 112. At the stage of delivery of FIG. 4A, the stimulation needle 110 has been percutaneously inserted (e.g., trans-perennially) and operated to confirm location of the tip 124 at the target site T. For example, following initial insertion of the needle body 122 to locate the tip 124 at an estimated position of the target site T, a response of the patient is monitored to determine/confirm a desired position of the tip 124. In some embodiments, the stimulation needle 110 can be operated (e.g., stimulation energy from a source (not shown) delivered to the tip 124) and a response by the patient monitored to identify the desired location of the target site T relative to the tip 124. For example, a response of one or more of sensed urethral pressure, sensed electrical nerve activity (ENG), visual observations of the muscles of the pelvic floor, sensed electromyography (EMG), patient sensory response, visual observations of eternal sphincter contraction (via optical scope, camera, or ultrasound image), etc., can be monitored to determine when the target site T has been “captured” by stimulation energy delivered at the tip 124.

The guidewire 112 may or may not be loaded within the stimulation needle 110 during operation of the stimulation needle 110 to obtain/confirm a desired location of the tip 124 relative to the target site T. Regardless, once acceptable stimulation capture has been identified, the guidewire 112 positioned within the stimulation needle 110 such that a distal end 130 (hidden in FIG. 4A but shown, for example, in FIG. 4B) of the guidewire 112 is located at the tip 124. The stimulation needle 110 is then retracted over the guidewire 112, with the guidewire 112 remaining in place as shown in FIG. 4B.

With reference to FIG. 4C, an introducer 140 is then inserted over the guidewire 112 and advanced. The introducer 140 can assume various forms known in the art, and is generally configured to expand contacted tissue in an atraumatic manner. In some embodiments, a spacer 142 (e.g., akin to an inner sleeve) can be provided with, or as an auxiliary component to, the introducer 140 that promotes sliding of the introducer 140 along/over the guidewire 112. Regardless, the introducer 140 is arranged relative to the guidewire 112 such that a leading end 144 of the introducer 140 is aligned with the distal end 130 (FIG. 4B) of the guidewire 112. The guidewire 112 (and, where provided, the spacer 142) can then be withdrawn from the introducer 140, resulting in the arrangement of FIG. 4D.

While maintaining a position of the introducer 140 relative to the target site T, a lead 160 is then advanced through the introducer 140 as shown in FIG. 4E. As a distal region 162 of the lead 160 exits the leading end 144 of the introducer 140 (best seen in FIG. 4F), one or more engagement features 164 (e.g., tine(s) such as self-deploying tines or other tissue-engaging bodies as known in the art) are deployed. The deployed engagement feature(s) 164 attach to tissue of the patient, thus capturing the distal region 162 relative to the patient. Because the leading end 144 of the introducer 140 is positioned at a known or desired location relative to the target site T, stimulation element(s) 166 (e.g., electrode(s)) provided with the distal region 162 will also be positioned at a known or desired location relative to the target site T with upon deployment of the engagement features 164. The introducer 140 can then be removed or withdrawn from over the lead 160, with the stimulation element(s) 166 of the so-placed lead 160 positioned to “capture” the nerve of the target site T (i.e., electrical energy emitted from the stimulation element(s) 166 at programmed levels will affect the nerve of the target site T in a desired manner).

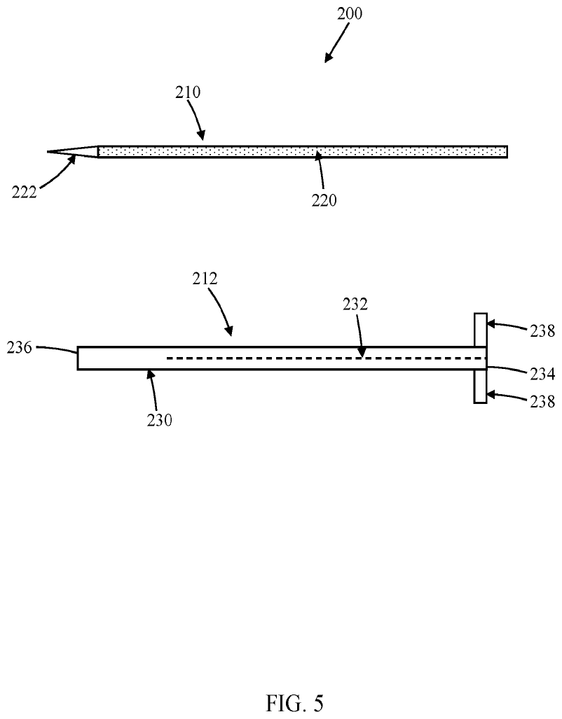

While the delivery system 100 has been described as including the stimulation needle 110 (FIG. 4A) that otherwise forms the lumen through which other components are received, other configurations are also acceptable. For example, FIG. 5 illustrates portions of another delivery system 200 in accordance with principles of the present disclosure and useful for placing a lead, such as a stimulation lead. The delivery system 200 includes a test needle or stimulation needle 210 and a sheath (such as a tear-away or split sheath) 212. The test needle 210 has a solid shaft 220 terminating at a tip 222. In other respects, the test needle 210 can be akin to the stimulation needle 110 (FIG. 4A) described above, generally formatted for piercing through tissue of the patient and delivering applied stimulation energy at the tip 220. In this regard, the shaft 220 can be formed of an electrically conductive material, with portions of an exterior of the shaft 220 apart from the tip 220 coated with an electrical insulator material, for example a polymer such as polyimide. With these and related embodiments, the exposed tip 220 serves as a conductor when electrical energy is applied to the test needle 210. Various other features can be provided with, or used with, the test needle 210, such as a handle, electrical contacts (for coupling to an energy source), etc.

The sheath 212 can assume various forms as is known in the art, and generally includes a sheath body 230 defining a lumen (hidden) sized to slidably receive at least the shaft 220 of the test needle 210. With optional embodiments in which the sheath 212 has a tear-away configuration, the tear-away sheath 212 can further include or form one or more features adapted to facilitate convenient splitting of the sheath body 230 by a user. For example, tear line(s) 232 (e.g., series of spaced apart perforations through a wall thickness of the sheath body 230) can be formed, extending from a proximal end 234 toward or to a distal end 236. Opposing tabs 238 are optionally provided at or near the proximal end 234, configured for convenient grasping by a user. With these and related embodiments, the tabs 238 are grasped and pulled away from one another, causing the sheath body 230 to split at the tear lines 232 into two (or more) separate portions. In other embodiments, the sheath 212 need not have a tear-away or split-type construction.

In some embodiments, the delivery system 200 is well-suited for deploying a stimulation lead for applying stimulating energy to a deep perineal branch of the pudendal nerve. During use, the test needle 210 percutaneously inserted (e.g., trans-perennially) and operated to confirm location of the tip 220 relative to an intended target site (e.g., a nerve). For example, following initial insertion of the test needle 210 to locate the tip 220 at an estimated position of the target site, a response of the patient is monitored to determine/confirm a desired position of the tip 220. In some embodiments, the test needle 210 can be operated (e.g., stimulation energy from a source (not shown) delivered to the tip 220) and a response by the patient monitored to identify the desired location of the target site relative to the tip 220. For example, a response of one or more of sensed urethral pressure, sensed electrical nerve activity (ENG), visual observations of the muscles of the pelvic floor, sensed electromyography (EMG), patient sensory response, visual observations of external sphincter contraction (via optical scope, camera, or ultrasound image), etc., can be monitored to determine when the target site has been “captured” by stimulation energy delivered at the tip 220.

Following insertion of the test needle 210 and successful location of the target nerve (or other target site), the sheath 212 is inserted over the test needle 210. In some embodiments, the sheath 212 can be loaded to the test needle 210 prior to insertion of the test needle 210 into the patient. Regardless, the sheath 212 is advanced over the test needle 210 so as to locate the distal end 236 in close proximity to the tip 220. Depth assessment of the sheath distal end 236 can be accomplished in various fashions. For example, depth assessment can be made via markings along a length of the test needle 210. While maintaining the sheath 212 in this location, the test needle 210 can then be removed. A small diameter stimulation lead (not shown) can then be inserted through the sheath 212, followed by removal of the sheath 212. For example, where the sheath 212 has the optional tear-away or split-type construction, the sheath 212 can be removed via the splitting operation described above. The optional tear-away configuration can promote removal of the sheath 212 from a lead otherwise having an enlarged hub and/or wires, from a non-isodiametric lead, a lead that is otherwise permanently fixed to a pulse generator, etc. With other lead configurations, the sheath 212 need not have a tear-away or split-type construction. In other embodiments, a guidewire (not shown) can be introduced into the sheath 212, after which the sheath 212 can be exchanged for a dilator (not shown). The dilator can then be used to introduce the lead. With these and related embodiments, depth assessment of the dilator relative to the sheath 212 can be accomplished in various manners, for example via markings along the length of the sheath 212.

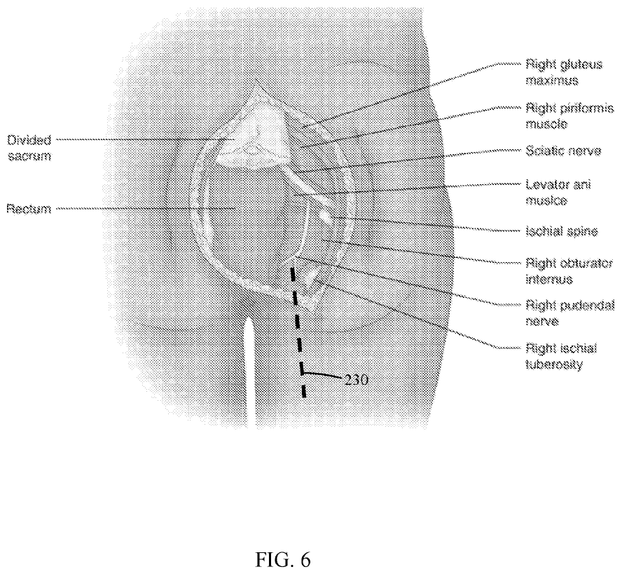

In other embodiments, systems and methods of present disclosure entail an open surgical approach for accessing a desired target site and placing a lead (or similar device) such that stimulating elements/electrodes of the lead are positioned at the target site, for example at or along the pudendal nerve. With reference to FIG. 6, in one example method of the present disclosure, a cut down is performed inferior to the gluteus maximus into the ischiorectal fossa (or ischioanal fossa) to locate the pudendal nerve or other target site. The term “cut down” is in reference to surgical access through the skin to a target site, akin to a dissection procedure. A small incision to help introduce a surgical tool or trocar or the like may be necessary. One example of an open surgical approach via the ischiorectal fossa to the pudendal nerve at the entrance to Alcock's canal is generally identified in FIG. 6 by a dashed line 230. As a point of reference, the ischiorectal fossa is a fat-filled, wedge-shaped space located lateral to the anal canal and inferior to the pelvic diaphragm. Other, similar cut down paths through the ischiorectal fossa could be employed to access other target nerve sites, for example deep perineal nerve branches. Once accessed, a cuff or similar electrode-carrying body of a lead (not shown) can be implanted to the target nerve (or other target site).

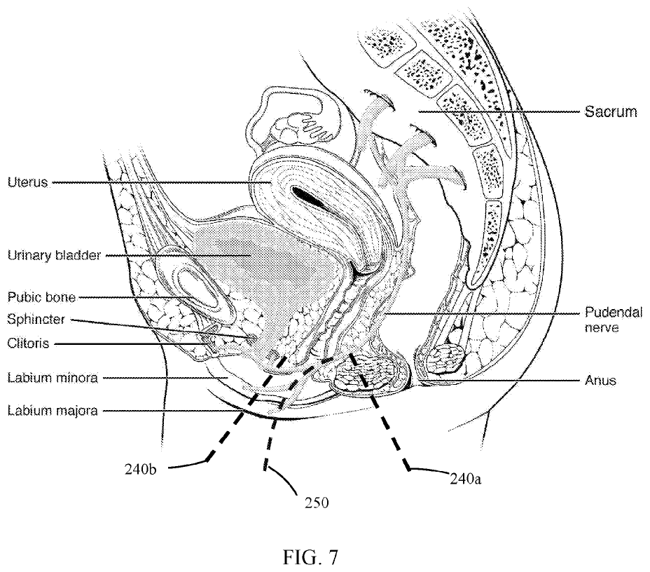

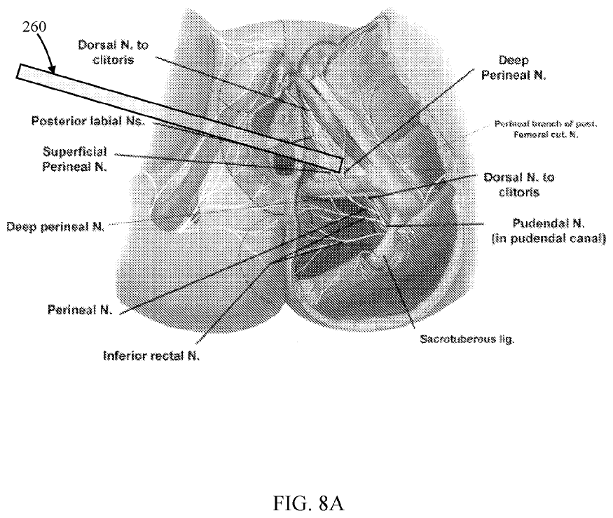

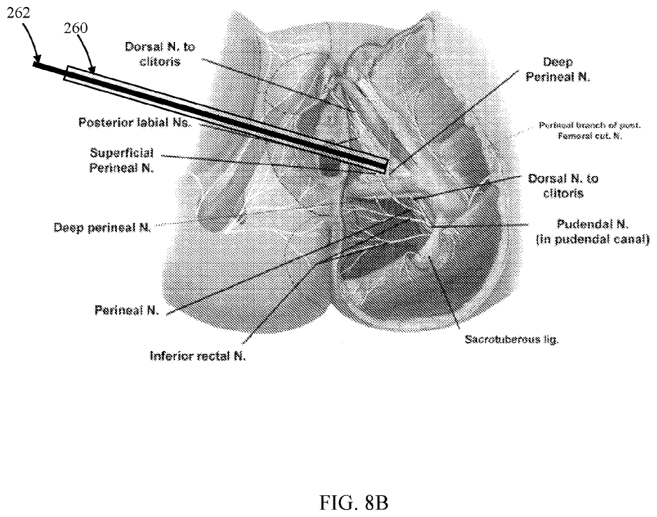

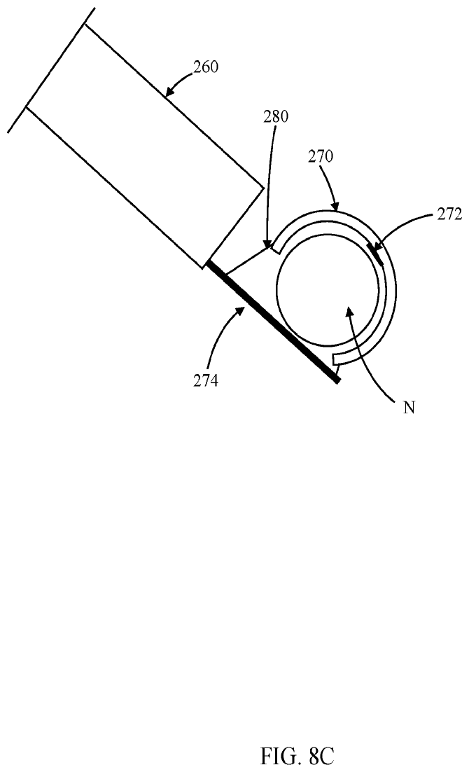

With reference to FIG. 7, in another example open surgical approach of the present disclosure, a transperineal cut down (e.g., with the patient in the lithotomy position not otherwise reflected by FIG. 7) is performed to locate the pudendal nerve or other target site. Two examples of the transperineal open surgical approach are generally identified in FIG. 7 by dashed lines 240a, 240b. The approach 240b is via a location near the urethra, but out of a plane of the view of FIG. 8. The incision can desirably be minimized (e.g., on the order of 3-6 centimeters). Once accessed, a cuff or similar electrode-carrying body of a lead (not shown) can be implanted to the pudendal nerve (or other target site) through the cut down. In another example open surgical approach of the present disclosure, a transvaginal cut down is performed to locate the pudendal nerve or other target site. An example transvaginal open surgical approach is generally identified in FIG. 7 by a dashed line 250 (it being understood that in the view of FIG. 7, the patient is not in the lithotomy position; the lithotomy position will provide more direct transvaginal cut down access to the pudendal nerve). Once accessed, a cuff or similar electrode-carrying body of a lead (not shown) can be implanted onto or adjacent to the pudendal nerve (or other target site) through the cut down.

With other stimulation lead placement methods of the present disclosure, laparoscopic-type instruments can be employed to place and implant a lead, and the electrodes carried thereby, relative to an intended target site, for example a deep perineal branch of the pudendal nerve, a dorsal genital nerve, a hypogastric nerve, a splanchnic nerve, etc. One example method is generally reflected by FIG. 8A, and includes inserting a laparoscope 260 via a transvaginal approach (with the patient in the lithotomy position) to provide access to the deep perineal branch of the pudendal nerve (or other target site). Subsequently, and as shown in FIG. 8B, a temporary stimulation probe 262 of a type known in the art can be inserted through the laparoscope 260 and operated to assist the clinician to locate a desired location along the targeted nerve (or nerve branch). Once the desired target site has been identified, a lead (not shown) can be delivered through the laparoscope and implanted relative to the target site. In one non-limiting example represented by FIG. 8C, a cuff body 270, provided as part of a lead and carrying at least one stimulation element or electrode 272, is advanced through the laparoscope 260 via a tool 274 (shown in highly simplified form). The tool 274 is operated to hold and locate opposite ends of the cuff body 270 above and below the target nerve N as shown. The tool 274 is then operated to release or deploy the cuff body 270, allowing the cuff body 270 to self-close or snap about the nerve N. The cuff body 270 and/or tool 274 can incorporate various features that facilitate delivery and placement about the nerve N, and one or more additional tools can be utilized. In one non-limiting example, the cuff body 270 can include or carry a tab or tail 280 biased to wrap against the cuff body 270. With these and related embodiments, a first tool can be manipulated to initially arrange the tab 280 in an extended state (relative to the cuff body 270) and wrap the tail about the nerve N on one side thereof. A gripper tool (akin to the tool 274) is then manipulated to pull the tab 280 back to an opposite side of the nerve N; when the tab 280 is subsequently released, the tab 280 returns toward the cuff body 270, operating to tow the cuff body 270 into position about the nerve N.

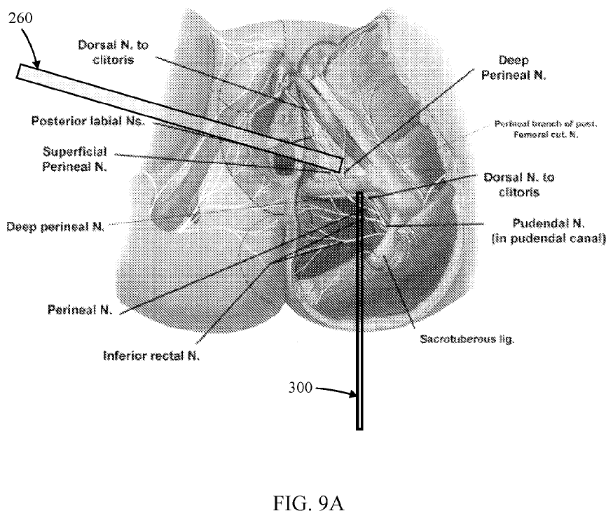

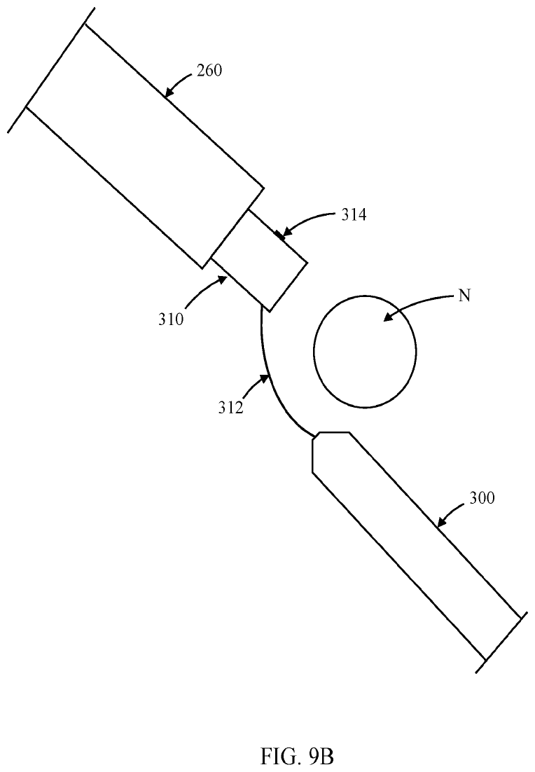

In yet other embodiments of the present disclosure, the transvaginally inserted laparoscope 260 arrangement of FIG. 8A can be employed to deliver and place a neurostimulator or IPG, such as a miniature neurostimulator. With these and related embodiments, and with reference to FIG. 9A, a separate incision (apart from the laparoscope 260) can be made and through which a grasping tool 300 is inserted (either directly, or via a delivery tube placed through the incision). As shown in FIG. 9B, a miniature IPG 310 is loaded into the laparoscope 260, and includes or carries a tow strap 312. The grasping tool 300 is operated to engage the tow strap 312 and pull the IPG 310 from the laparoscope 260, deploying the IPG 310 from the arrangement of FIG. 9B to a desired position relative to the target nerve N (e.g., pudendal nerve, deep perineal nerve branch, hypogastric nerve, splanchnic nerve, etc.). For example, the IPG 310 can be arranged by the tow strap 312 such that a stimulation element or electrode 314 provided with the IPG 310 is proximate the target nerve N. Alternatively, where the IPG 310 is formatted for electrical connection to a separately-provided lead, the lead can be arranged relative to the target nerve N in advance of delivering the IPG 310 (e.g., the cuff body 270 and related techniques described above with respect to FIG. 8C). Once the lead has been positioned, the IPG 310 can be towed into position as described above. Regardless, the IPG 310 can be fixed to native tissue in various fashions, for example via a mesh pouch around the IPG 310, suture anchor loops (or similar features with the IPG 310), passive tines, etc.

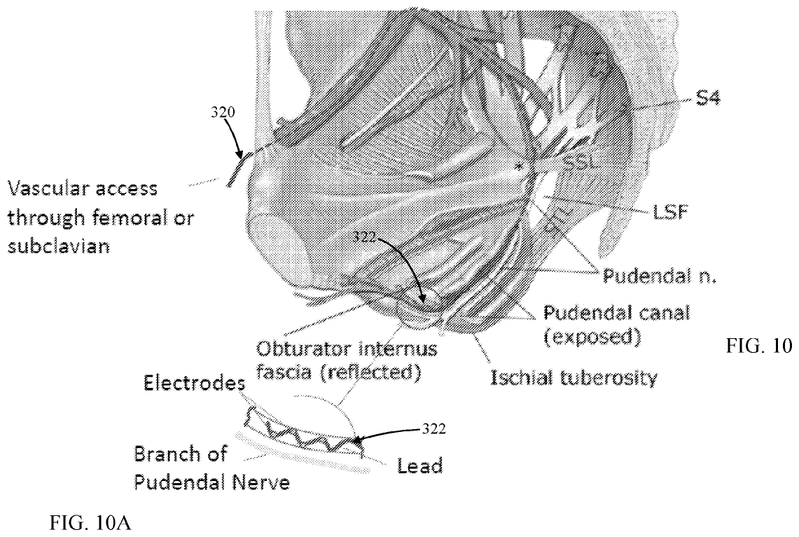

Other stimulation lead placement methods of the present disclosure entail endoluminal techniques. For example, as shown in FIG. 10, with some methods, a stimulation lead 320 is introduced endoluminally through a transcutaneous access into the femoral vein. The lead 320 can be of a type known in the art, and is generally configured for articulation about a tortuous path of the vasculature (e.g., the lead 320 is provided with steering capabilities such as via a bent tip stylet, catheter steering techniques, pull wire(s), etc.). The lead 320 is directed or navigated through the vasculature with the aid of imaging techniques such as fluoroscopy, ultrasonography, etc. Optionally, contrast dye can be injected to further aid in visualizing the vasculature and identifying desired or targeted nerve branching.

As a distal region 322 of the lead 320 has been advanced along the femoral vein to a location generally proximate the target nerve (e.g., pudendal nerve, splanchnic nerve, hypogastric nerve, etc.), the lead 320 is manipulated to locate the distal region 322 in concomitant vessel(s) extending from the femoral vein to a location more proximate the target nerve. The lead 320 is then further advanced to locate the distal region 322 (and thus stimulation element(s) or electrode(s) carried thereby) adjacent to the target nerve. The lead 320 is then operated to deliver test stimulation(s), allowing the clinician to confirm that the attained location is appropriate for capturing the target nerve. Upon confirming desired placement of the distal region 322, the lead 320 can be anchored relative to the vasculature. For example, the lead 320 can be anchored to the patient at the vasculature access location. Alternatively or in addition, fixation can be provided at or proximate the distal region 322. In some non-limiting examples reflected by the view of FIG. 10A, at least the distal region 322 of the lead 320 can be formed to naturally assume a serpentine or serpentine-like shape that provides a frictional interface/fixation with the vascular wall. With these and related embodiments, the serpentine shape can be rendered straight via insertion of a stylet or similar tool, with the distal region 322 self-returning to the serpentine shape upon removal of the stylet. Alternatively or in addition, fixation of the distal region 322 can be accomplished via tines (e.g., self-deploying tines) or similar bodies or mechanisms. Regardless, following securement of the lead 320, other components of the treatment system, such as an IPG (not shown) can then be surgically placed.

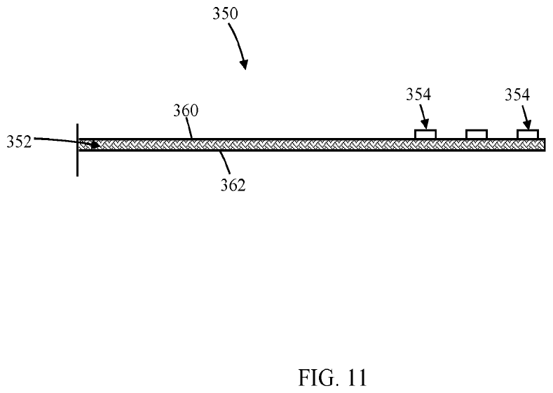

Yet other embodiments of the present disclosure relate to mesh-ribbon type leads and corresponding implantation tools and techniques. As a point of reference, FIG. 11 illustrates, in simplified form, portions of an example of an implantable mesh-ribbon type lead 350. The lead 350 generally includes a mesh body 352 carrying one or more electrodes 354. The mesh body 352 is formed of a biocompatible material (e.g., metal wire, woven polymer textile mesh), and can be relatively flat. For example, opposing major surfaces 360, 362 of the mesh body 352 are substantially flat or planar (i.e., within 10% of a truly planar surface); in other embodiments, the mesh body 352 can curve or conform to the shape of a surface to which it is applied. With the mesh format, the mesh body 352, and thus the lead 350, is flexible and extensible. The electrodes 354 can be arranged along the mesh body 352 in various fashions (e.g., linearly aligned, equidistantly-spaced, patterned, etc.) with an emitting face of each of the electrodes 354 being exposed relative to the first major surface 360. While the electrodes 354 are shown as been secured to the first major surface 360, in other embodiments, one or more or all of the electrodes 354 can be partially embedded into a thickness of the mesh body 352. Regardless, insulated wiring (not shown) is electrically connected to each of the electrodes 354 and extends through the mesh body 352. The mesh-ribbon type lead 350 can be utilized with various end use applications, such as sensing EMG, delivering stimulation therapy, etc. In some embodiments, the mesh-ribbon type lead 350 is well-suited for end use applications benefitting from a flexible and extensible flat lead, for example to be located in the interfacial plane just adjacent to a muscle. The mesh body 352 can provide for improved fixation of the lead 350 via tissue encapsulation over time (the mesh body 352 provides pathways for tissue to grow through).

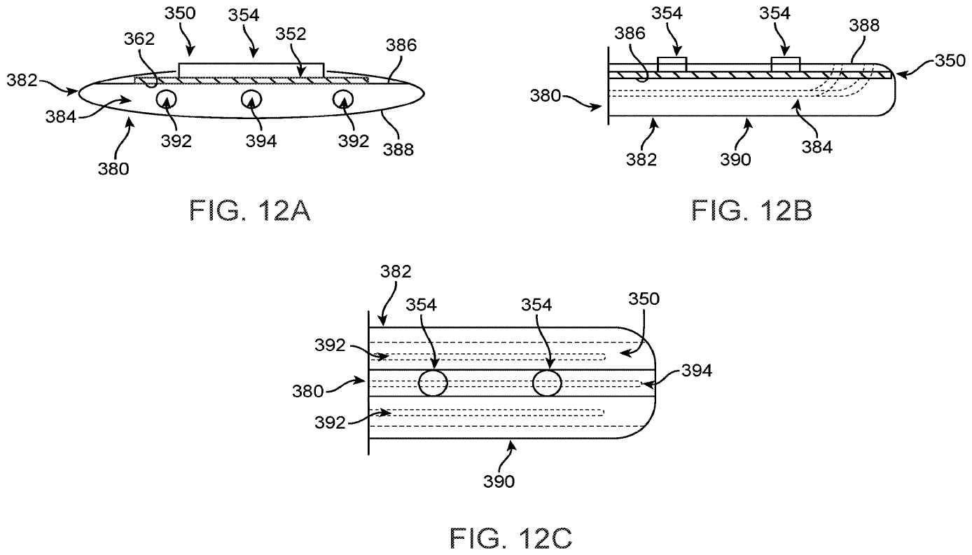

The mesh-ribbon type lead 350 can be implanted in various manners. FIGS. 12A-12C illustrate one embodiment of an introducer tool 380 of the present disclosure that can be useful for delivering/implanting the lead 350 (as a point of reference, the lead 350 is loaded to the introducer tool 380 in the views of FIGS. 12A-12C). The introducer tool 380 includes an elongated introducer body 382 including or defining a base 384. The base 384 forms or defines a receiving face 386 opposite an exterior face 388. The receiving face 386 is flat or planar, configured to receive and support the second major surface 362 (best seen in FIG. 11) of the mesh body 352. The exterior face 388 can have various shapes or attributes conducive to atraumatic contact with tissue (e.g., the exterior face 388 can be generally curved or otherwise free of sharp corners). Along at least a distal segment 390 of the introducer body 382, the receiving face 386 is open or exposed. That is to say, the receiving face 386 may or may not be exposed proximal the distal segment 390 (e.g., the view of FIG. 12A generally reflects that the exterior face 388 can extend “over” the receiving face 388 along portions of the introducer 380), at least along the distal segment 390, the receiving face 386 is open to the external environment.

With the above construction, the lead 350 can be loaded to the introducer body 382, with the second major surface 362 of the mesh body 352 abutting or residing on the receiving face 386. An arrangement of the lead 350 relative to the introducer body 382 in the loaded state is such that the electrodes 354 are positioned along the distal segment 390. With this arrangement, and commensurate with the descriptions above, the electrodes 354 are thus exposed or open to the external environment of the introducer body 382. As a point of reference, while the electrodes 354 are shown as being raised or projecting beyond the introducer body 382, in other embodiments, the electrodes 354 can be flush or substantially flush with the exterior face 388. Portions of the lead 350 proximal the distal segment 390 (and thus portions of the lead 350 proximal the electrodes 354) can be more completely confined within the introducer body 382. Regardless, the lead 350 can be secured to the introducer body 382 (in the loaded state) in various manners. In some embodiments, the introducer tool 380 can include one or more tines (not shown) carried by the introducer body 382. The tines can be extended/retracted relative to the introducer body 382 via holes or passages 392 formed through a thickness of the base 384. When extended beyond the corresponding hole 392, the tine(s) readily interface with and capture the mesh body 352. To deploy the lead 350, the tines can be retracted from engagement with the mesh body 352.

During use, with the lead 350 loaded to the introducer tool 380 as described above, the introducer tool 380 is manipulated to locate the electrodes 354 proximate a target site (e.g., targeted nerve). The lead 350 can then be operated to deliver test stimulation via the electrodes 354 to confirm a desired placement relative to the target site. In this regard, because the electrodes 354 are exposed relative to the introducer body 382 in the loaded state, test stimulation energy can be delivered followed by re-locating the introducer tool 380 (and thus the lead 350) as desired. Temporary fixation of the lead 350 relative to the patient's anatomy can be provided in various manners. In some examples, one or more shape memory metal wires (not shown) can be provided. The wire(s) can be configured to self-assume a spiral shape, and can be rendered straight when loaded through a passageway 394 formed in the introducer body 382. When deployed from the introducer body 382, the wire(s) self-revert to the coil shape, achieving a frictional interface with surrounding tissue or otherwise anchoring the lead in place with respect to the tissue.

Once a desired location of the lead 350 has been attained, the lead 350 can be deployed from the introducer tool 380, for example by retracting the tines from the mesh body 352 as described above and then retracting the introducer tool. In some embodiments, fixation of the lead 350 to the patient's anatomy can be facilitated by delivering metal clips or sutures through the passageway 394. The so-delivered clips or similar bodies deploy through the mesh body 352, and secure to surrounding tissue.

Returning to FIG. 11, the lead 350 (including the mesh body 352) can be delivered and implanted in other manners that may or may not entail the introducer tool 380 (FIG. 12A). For example, in some embodiments, the mesh body 352 can have a tube-like construction, defining a lumen. A stiffening paddle or similar tool can be inserted into the lumen prior to delivery into the patient. The so-supported lead 350 can then more readily be directed to a target site. Once placed at a desired location, the stiffening tool can be removed from the mesh body 352, so that the lead 350, upon final implant, is highly flexible and more likely to stay in place.

Various systems and methods of the present disclosure have been described as, at least in part, a clinician determining whether a stimulation element (e.g., carried by or as part of a lead, stimulation needle, test needle, etc.) has been desirably located relative to a nerve (e.g., a target nerve) such that stimulation energy delivered from the stimulation element appropriately “captures” the nerve (e.g., the nerve responds in a desired manner to the applied stimulation energy). This can be accomplished using techniques known in the art. Known techniques entail the clinician positioning the stimulation element at a best guess location, manually prompting delivery of stimulation energy, manually assessing the patient's response to the applied stimulation energy, and making a best guess as to whether or not the location of the stimulation element is acceptable. Under circumstances where the clinician decides that the location is not acceptable, the clinician then manually re-positions the stimulation element to another “best guess” location and the steps are repeated.

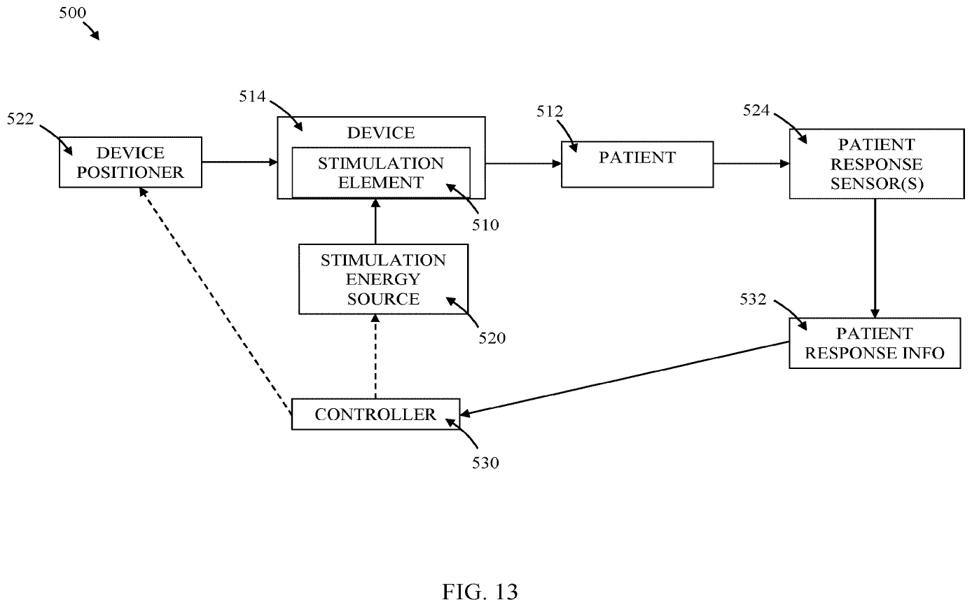

With the above in mind, other aspects of the present disclosure relate to closed loop systems and methods for assisting and/or optimizing stimulation element placement within a patient. The closed loop assistance systems and methods can be used with any of the systems and methods described above (e.g., where reference is made above to a clinician seeking to confirm nerve “capture”), as well as with any other system or method in which optimized stimulation element placement is desired, for example to improve patient response to therapy, decrease unwanted extraneous stimulation, decrease reliance on nuanced skill of clinical staff, minimized complications, decrease surgery time, etc. With this in mind, one example of a closed loop system and method 500 for assisting in locating a stimulation element 510 within a patient 512 is diagrammatically represented in FIG. 13. As a point of reference, the stimulation element 510 can be provided with or carried by a device 514 that can assume a wide variety of forms known in the art configured for placement within the human body and for delivering stimulation energy to the stimulation element 510, such as a lead, test needle, stimulation needle, test probe, etc. The device 514 may carry or include two or more stimulation elements. Moreover, a variety of techniques can be used to introduce the device 514, and thus the stimulation element(s) 510 carried thereby, into the patient 512, typically specific to the particular target nerve (or other anatomy) of interest and a format of the device 514 (e.g., any of the introduction techniques of the present disclosure).

A stimulation energy source 520 is electrically coupled to the stimulation element 510 via the device 514, and is operable to deliver energy to the stimulation element 510. In general terms, the energy source 520 can be of a type known in the art configured for use with the device 514, and can provide various features for controlling a format or parameters of delivered energy (e.g., amplitude, frequency, pulse rate, pulse width, pulse train length, etc.). A position of the device 514, and thus of the stimulation element 510 carried thereby, relative to the patient 510 is controlled or dictated by a device positioner 522. The device positioner 522 can assume various forms. In some embodiments, the device positioner 522 is a human (e.g., surgeon or other clinician). In other embodiments, the device positioner 522 can be partially or fully automated (e.g., a surgical robot as known in the art). One or more sensors 524 are provided, generally configured to detect or report information indicative of a response of the patient 512 to stimulation energy applied by the stimulation element 510. A wide variety of sensor(s) 524 can be employed, typically as a function of an expected patient response (or patient response of interest) to applied stimulation. For example, the sensor(s) 524 can be located within the patient 510, or external. By way of non-limiting example, some useful sensor formats can include electrical nerve activity (ENG) sensors, electromyography (EMG) sensors, pressure sensors, accelerometers (or similar motion-detecting formats), visual sensors (e.g., visual or machine detection of patient movement generally and/or movement of a particular anatomical feature of the patient), strain gauges, etc. In yet other embodiments, the sensor(s) 524 can include an input device through which a clinician can electrically note visual observations of a patient's response.

The systems and methods 500 can further include a controller 530. The controller 530 can assume a variety of forms, and can assume any of the formats described above in reference to a “controller” or a “control portion”. The controller 530 is programmed to electronically receive and act upon information or data collected or obtained by the sensor(s) 524, represented diagrammatically in FIG. 13 at 532.

In some embodiments, the controller 530 can be provided apart from the energy source 520. With these and related embodiments, the controller 530 can be electronically connected to the energy source 520 and is programmed to dictate or prompt operation of the energy source 520, for example automatically prompting the energy source 520 to generate/deliver stimulation energy to the stimulation element 510 at determined times and/or in a determined format. In other embodiments in which the controller 530 is provided apart from the energy source 520, the controller 530 can be programmed to inform a user (e.g., clinician) of determined stimulation energy delivery timing and/or format via a display or the like, with the user then following these instructions to manually operate the energy source 520. In yet other embodiments, the controller 530 can be an integrated component of the energy source 520 (e.g., the energy source 520 can include a controller or control portion that dictates operation (e.g., delivery of energy to the stimulation element 510) and that is programmed to perform the operations described below).

The controller 530 can optionally be programmed to determine adjustments to a location of the device 514, and thus of the stimulation element 510, relative to the patient 512 as described below. With these and related embodiments, the controller 530 can be programmed to inform a user (e.g., clinician) of a determined position adjustment, with the user then following these instructions to re-position the device 514 relative to the patient (e.g., where the device positioner 522 is a human that manually manipulates the device 514; where the device positioner 522 is robot-based and is configured for a user to enter or select a desired position or movement to be implemented by the device positioner 522; etc.). With other embodiments in which the device positioner 522 is a robot-based device or the like and is configured to electronically receive operational instructions or prompts, the controller 530 can be electronically connected to the device positioner 522 and programmed to dictate or prompt operation of the device positioner 522 to automatically move the device 514 in a determined fashion. In yet other embodiments, the controller 530 can be an integrated component of a robotic device positioner 522.

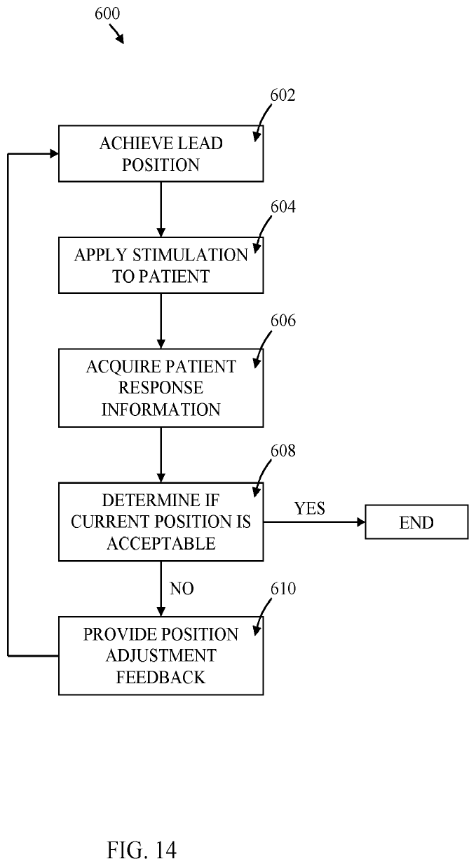

FIG. 14 illustrates a method 600 in accordance with principles of the present disclosure for closed loop assistance in placing or implanting a lead (that includes or carries at least one stimulation element) within a patient and available, for example, via the system 500. While the method 600 references a lead, other stimulation element-carrying devices (e.g., test probe, stimulation needle, etc.) can alternatively be employed. With cross-reference between FIGS. 13 and 14, at 602, a position of the lead 514, and in particular the stimulation element 510, within the patient 512 is achieved. At 604, the energy source 520 is operated to deliver stimulation energy to the stimulation element 510 that in turn applies stimulation energy to the patient 512. At 606, patient response information 532 is acquired via the sensor(s) 524, indicative of the patient's response to the applied stimulation energy. The patient response information 532 is reviewed, for example by the controller 530, to determine whether a current position of the stimulation element 510, and thus of the lead 514, relative to the patient 512 is acceptable at 608. Various rules, protocols, algorithms, etc., relevant to the particular procedure (and desired stimulation energy effect to the patient upon final implant) can be utilized by, or programmed to, the controller 530 for automatically assessing the patient response information 532. Under circumstances where the current position of the stimulation element 510 is not deemed as being acceptable (“no” at 608), position adjustment feedback is provided at 610. The position adjustment feedback can assume various forms, and can include guidance or a recommendation for re-positioning the lead 514, and thus the stimulation element 510. Various rules, protocols, algorithms, etc., can be utilized by, or programmed to, the controller 530 for automatically determining a desired new position of the stimulation element 510 relative to the patient 512, for example based upon or with reference to patient response information obtained at previous positions of the simulation element 510. The method 600 then returns to 602 and the steps repeated until an acceptable position of the stimulation element 510 is obtained.

In some optional examples, the automated lead placement systems and methods of the present disclosure can be configured to indicate to the clinician that a first placed unilateral lead is adequate for expected therapy delivery. Alternatively or in addition, the systems and methods can be configured to indicate to the clinician that a second lead is necessary in order to better attain an effective therapy (e.g., that the unilateral lead alone is not adequate and that a second (or more) lead should be implanted to provide bilateral stimulation, multiple ipsilateral leads, etc.).

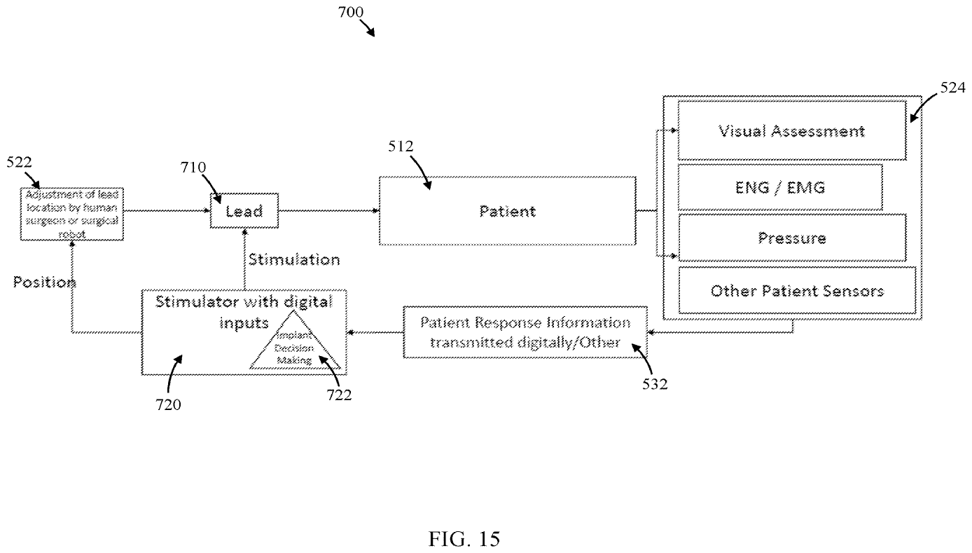

Another example of a closed loop system and method 700 for assisting in locating a stimulation element, as carried by a lead or similar device 710, within the patient 512 is diagrammatically represented in FIG. 15. A stimulator 720 is electrically coupled to the lead 710 and operates to deliver stimulation energy (“Stimulation” in FIG. 15) to the lead 710 (and is thus akin to the stimulation energy source 520 of FIG. 13). The stimulator 720 can be provided with digital inputs for receiving the patient response information 532 generated or obtained by the sensor(s) 524. The stimulator 720 is further illustrated as operating a controller or control portion that is programmed to perform implant decision making 722. Examples of the protocols or algorithms operated by, or programmed to, the implant decision making 722 are provided below. With the example of FIG. 15, the sensor(s) 524 can include one or more of a visual assessment device, an ENG or EMG sensor, a pressure sensor, etc. Finally, the closed loop system and method 700 includes the device positioner 522 as described above, and can be a human operator (e.g., human surgeon or clinician) or a surgical robot.

In some examples, the system and method 700 can include the stimulator 720 informing one or more clinicians (e.g., surgeon, operating room staff member, etc.) of the status of the patient response information 532 while automatically scrolling through a series of stimulation parameters while a position of the lead 710 is adjusted by the device positioner 522 (e.g., human surgeon or surgical robot). As the patient response information 532 is detected, stimulation parameters can be adjusted by the stimulator 720. Optimal location of the lead 710 relative to the patient 512 can be determined based upon prioritization of various patient response information factors. For example, where the lead 710 is intended to operate to treat urinary incontinence, EUS pressure response information, internal sphincter pressure response information, or a combination of pressure can be deemed a key parameter.

In other examples, the stimulator 720 can be an intraoperative stimulator programmed to manually or automatically notify the treating clinician(s) (e.g., surgeon, operating room staff member, etc.) of the status of any patient response implicated by the patient response information 532. Notification can, in some embodiments, happen visually from a screen on or connected to the intraoperative stimulator 720, lights on the intraoperative stimulator 720, haptic feedback from the intraoperative stimulator 720 provided to the treating clinician(s) via a worn strap, via audio emitted from the intraoperative stimulator 720, etc. In some embodiments, the intraoperative stimulator 720 can be programmed to adjust parameters of applied stimulation continuously while the lead 710 is being placed. Stimulator parameters could include one or more of varying amplitude, frequency, pulse width, pulse train length, on/off periods, electrode configuration, etc. Pre-set routines or programs can be provided with (e.g., programmed to) the intraoperative stimulator 720 that, when executed, cycle though stimulation parameters while monitoring lead position (e.g., robot monitoring, manual monitoring, external imaging and localization on the lead 710, etc.). Based on initial feedback from the intraoperative stimulator 720, the pre-set programs can, in some embodiments, be adapted to minimize the time needed for assessment. For example, the programming can consider the patient response information 532 to adjust one or more stimulation parameters determined to be of interest by the system 700, all of which may assist with eliciting a patient response during lead placement. The adjustment of stimulation parameters could consider the patient response information 532 detected in order to assist lead location optimization. Furthermore, the system 700 can optionally include accessories that operate to manipulate the patient 512 automatically and/or with clinician guidance, such as an external abdominal belt that could temporarily increase intraabdominal pressure to help evaluate an applied stimulation format or setting against, for example, urethral leak point pressure, urethral pressure, etc. Other accessories can alternatively be employed. With these and related embodiments, feedback information can be provided that can implicate optimal stimulation parameters. Additionally, the automated system 700 can be programmed to pause and request the clinician to perform a procedure on the patient while the patient response information 532 is being collected, such as external TENS stimulation of the perineum or abdominal wall. Regardless, lead location can be adjusted manually by the clinician or automatically by a surgical robot electronically connected to the intraoperative stimulator 720. Lead location could be adjusted in all dimensions available. The clinician or robot can, in some embodiments, be instructed to remove and attempt reinsertion at a different location under various circumstances. Instruction for removal or adjustment can, in some embodiments, be informed based on a minimum acceptable patient response as decided by the intraoperative stimulator 720.

In some embodiments, following optimal lead placement, the intraoperative stimulator 720 can be programmed to run through a stimulation parameter optimization routine or cycle to determine optimal stimulation parameter setting(s) and/or to recommend take-home treatment programs for the patient 512 to utilize.

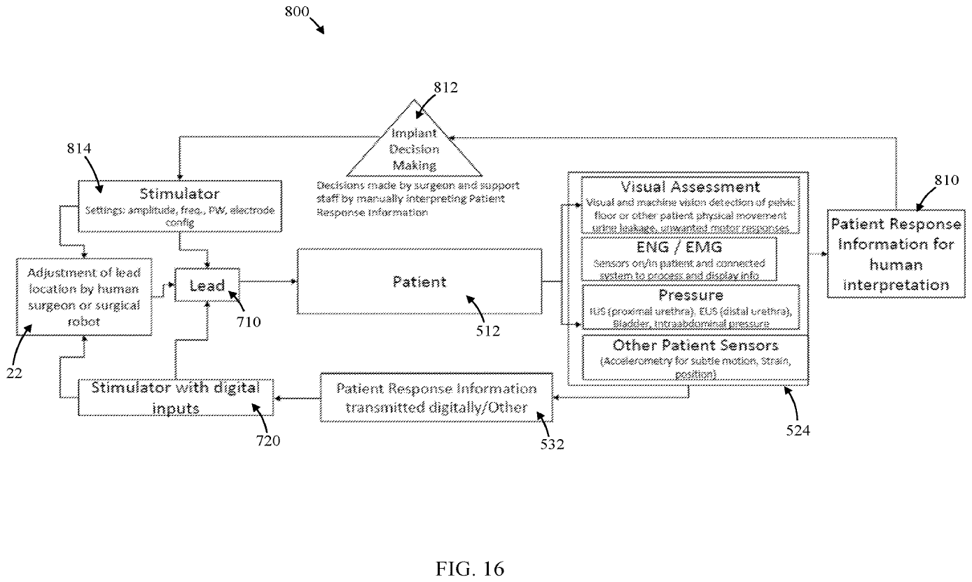

Another example of a closed loop system and method 800 for assisting in locating a stimulation element, as carried by the lead or similar device 710, within the patient 512 is diagrammatically represented in FIG. 16. In many respects, the system 800 is highly akin to the system 700 (FIG. 15) described above, and includes the intraoperative stimulator 720 programmed to automatically assist in placing the lead 710 and/or in determining optimal stimulation parameters based on the electronically-delivered patient response information 532. In addition, the system and corresponding methods 800 can facilitate or allow for data or information from the sensor(s) 524 to be provided to one or more clinicians, for example as patient response information for human interpretation (represented diagrammatically in FIG. 16 at 810). The so-provided patient response information 810 can be reviewed and forms the basis for clinician decision-making 812, for example to evaluate whether or not a current position of the lead 710 is acceptable and/or whether or not a format of applied stimulation is acceptable. Under circumstances where it is determined that one or both of the current position or the applied stimulation format is unacceptable, the clinician can then adjust a position of the lead 710 via the human or robotic device positioner 522 as desired, and effect application of stimulation energy via operation of a stimulator 814. In this regard, the clinician can operate the stimulator 814 to select a desired format of the applied stimulation energy, for example by varying one or more parameters such as amplitude, frequency, pulse width, electrode configuration, etc.

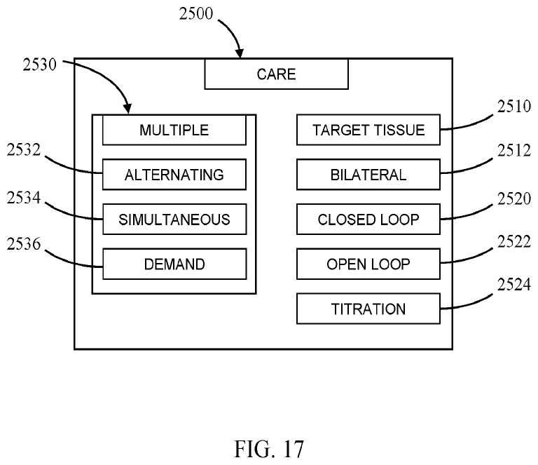

FIG. 17 is a block diagram schematically representing a care engine 2500 of a control portion. In some examples, the care engine 2500 may comprise an example implementation of, and/or at least some of substantially the same features and attributes as, any of the IPGs, care engines and/or the control portions (e.g., FIGS. 3-16) of the present disclosure. Accordingly, the various functions and parameters of the care engine 2500 may be implemented in a manner supportive of, and/or complementary with, the various functions, parameters, portions, etc., of any of the devices and control portions and/or various functions, parameters, portions, etc., relating to stimulation throughout examples of the present disclosure. In some examples, the care engine 2500 may include an implementation of the control portion 70 of FIG. 3.

In some examples, different target tissue may be stimulated using at least one stimulation element. The target tissues may be stimulated at the same time (e.g., simultaneously or overlapping times) or at different times and/or in response to different sensed parameters, such as those described and illustrated in connection with at least FIGS. 3-16.

In some examples, any of the methods, apparatuses, and/or devices may be used to provide bladder and/or bowel dysfunction care to different target tissue, including those described in connection with at least FIGS. 1-16.

As shown by FIG. 17, in some examples, via target tissue parameter 2510, stimulation may be delivered to select target tissue such as, but not limited to, the pudendal nerve, the pelvic nerve, the sacral nerve, hypogastric, or branches thereof. In some examples, target tissues may include any muscles which affect and/or promote continence (e.g., urethral sphincters, detrusor, etc.) and/or nerves which innervate such muscles. In some examples, target tissue includes a combination of nerves and/or muscles such as, but not limited to, terminal fiber ends of nerves where a nerve ending terminates into (or at) the muscle being innervated.

In some examples, in addition to or instead of selecting different tissue for stimulation, the target tissue parameter 2510 may comprise adjusting care parameters (e.g., stimulation parameters) via selecting between (or using a combination of) various locations along a nerve such as stimulating multiple different sites along a particular nerve.

In some examples, in addition to or instead of selecting different nerves for stimulation, the target tissue parameter 2510 may comprise adjusting care parameters via selecting between (or using a combination of) different fascicles within a particular nerve in order to selectively stimulate target efferent fibers while omitting (or minimally impacting) stimulation of other, non-target fibers and/or to selectively stimulate target efferent fibers while omitting (or minimally impacting) stimulation of other, non-target fibers.

In some examples, the care engine 2500 may implement stimulation according to a bilateral parameter 2512 in which stimulation is applied to target tissue on both sides (e.g., left and right) of the patient's body. In some such examples, the bilateral stimulation may be delivered to the same target tissue (e.g., pudendal nerve, pelvic nerve, sacral nerve, hypogastric, or branches thereof) on both sides of the body. However, in some examples, the bilateral stimulation may be delivered to different target tissue or tissue on a left side of the body while stimulating another nerve or tissue on a right side of the body, or vice-versa. It may be beneficial to deliver to one stimulation target site with one pulse train, while delivering a second pulse train to a second site where the second pulse train is delayed from the first pulse train. In addition, the frequencies and other stimulation parameters between the first and second (or additional) pulse trains and sites are different from each other.

In some examples, the bilateral parameter 2512 may be implemented in a manner complementary with the alternating parameter 2532, simultaneous parameter 2534, or demand parameter 2536 of multiple function 2530, as further described below.

In some examples, the care engine 2500 may comprise a multiple function 2530 by which various care parameters may be implemented in dynamic arrangements. In some such examples, the care engine 2500 may comprise an alternating parameter 2532 by which care provided to one target tissue (e.g., pudendal nerve) may be alternated with care provided to at least one other target tissue (e.g., pelvic nerve). However, the alternating parameter 2532 also may be applied in combination with the bilateral parameter 2512 to apply care to the target tissue (or different target tissue) on opposite sides of the body in which care may be applied on a left side of the body and then applied on the right side of the body in an alternating manner. As used herein, applying or providing care to target tissue may include applying stimulation and/or mechanically maneuvering the target tissue.

In some examples, the care engine 2500 may comprise a simultaneous parameter 2534 by which care may be applied simultaneously to at least two different target tissues. In some examples, the at least two different target tissues comprise two different tissues, such as the pudendal nerve and the pelvic nerve. In some examples, the at least two different target tissues may comprise two different locations along the same tissue or two different fascicles of the same nerve. In some examples, the simultaneous parameter 2534 may apply stimulation per bilateral parameter 2512 simultaneously on opposite sides of the body to the same tissue or different tissue, and/or apply mechanical maneuvering simultaneously on opposite sides of the body to the same tissue.

In some examples, the care engine 2500 may comprise a demand parameter 2536 by which care may be applied to at least one target tissue on a demand basis. For example, stimulation may be applied to one nerve (e.g., pudendal nerve, such as a deep perineal branch thereof) which may be sufficient to achieve the patient metric (e.g., continence) for most circumstances, but may become insufficient for some situations. In the latter situation, to achieve the target patient metric, via the demand parameter 2536, stimulation of a different nerve (e.g., pelvic nerve) may be implemented in addition to, or instead of, stimulation of the first nerve (e.g., pudendal nerve) which was previously being stimulated. In some examples, the first or primary nerve being stimulated may be a nerve other than the pudendal nerve.

In some examples, the care engine 2500 also may further implement at least some aspects of the control portion of FIGS. 3-16 and/or according to at least one of a closed loop parameter 2520, open loop parameter 2522, and nightly titration parameter 2524.

In some examples, the care engine 2500 comprises a closed loop parameter 2520 to deliver care based on sensed patient physiologic information and/or other information (e.g., environmental, temporal, captured by an external system and communicated to the care engine 2500, etc.). In some such examples, via the closed loop parameter 2520 the sensed information may be used to control the particular timing of the care according to bladder fullness information. In some such examples and as previously described, the bladder fullness information and/or other information used with the closed loop parameter 2520 may be determined via the sensors, devices, sensing portions, as previously described in association with at least FIGS. 3-16.

In some examples, the care engine 2500 comprises an open loop parameter (e.g., 2522 in FIG. 17) by which bladder and/or bowel dysfunction care (e.g., “use”) is applied without a feedback loop of sensed physiologic information. In some such examples, in an open loop mode the care is applied during a treatment period without (e.g., independent of) information sensed regarding the patient's bladder fullness, detrusor levels, etc.

In some examples, the care engine 2500 comprises a titration parameter 2524 by which an intensity of the bladder and/or bowel dysfunction therapy may be titrated (e.g., adjusted) to be more intense (e.g., higher stimulation amplitude, greater frequency, and/or greater pulse width) or to be less intense within a treatment period.

In some such examples, the titration parameter 2524 may be implemented according to at least some aspects of the example methods and/or example devices of FIGS. 3-16. Accordingly, in some examples, the titration parameter may be implemented as automatic titration, while in some examples, the titration parameter may be implemented via manual titration by a patient (or clinician), such as to adjust one or more stimulation parameters. In some examples, the titration parameter may be implemented via combination of patient/manual titration and automatic titration to guide the patient in a manner complementary with manual titration.

In some examples, at least some aspects of the titration parameter 2524 of the care engine 2500 and/or at least some aspects of titration as generally disclosed throughout FIGS. 3-16 in examples of the present disclosure may comprise (and/or may be implemented) in a manner complementary with and/or via at least some of substantially the same features and attributes as described in: (i) PCT Publication No. 2020/243104 (Rondoni, et al.), and (ii) PCT Publication No. WO 2022/192726 (Rondoni, et al.), each of which are hereby incorporated by reference in their entirety.

The various ranges provided herein include the stated range and any value or sub-range within the stated range. Furthermore, when “about” is utilized to describe a value, this includes, refers to, and/or encompasses variations (up to +/−10%) from the stated value.

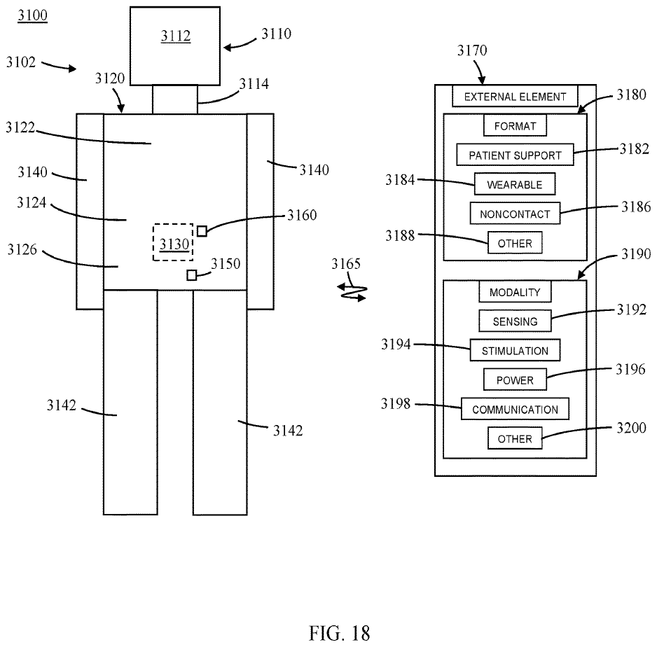

FIG. 18 is a block diagram schematically representing an example arrangement 3100 including patient's body 3102, including example target portions 3110-3142 at which at least some example sensing element(s) and/or stimulation elements may be employed to implement at least some examples of the present disclosure.

As shown in FIG. 18, patient's body 3102 comprises a head-and-neck portion 3110, including head 3112 and neck 3114. As further shown in FIG. 18, the patient's body 3102 comprises a torso 3120, which comprises various organs, muscles, nerves, other tissues, such as but not limited to those in pectoral region 3122 (e.g., lungs, cardiac), abdomen 3124, and/or pelvic region 3126. Organs, muscles, nerves, other tissues of the abdomen 3124 and/or the pelvic region 3126 include bladder 3130, urethra, anus, pelvic floor, etc. As further shown in FIG. 18, the patient's body 3102 comprises limbs such as arms 3140 and legs 3142.

It will be understood that various sensing elements (and/or stimulation elements) as described throughout the various examples of the present disclosure may be deployed within the various regions of the patient's body 3102 to sense and/or otherwise diagnose, monitor, treat various physiologic conditions such as, but not limited to the above-described examples in association with FIGS. 3-17. In some such examples, a stimulation element 3150 may be located in or near the pelvic region 3126 for treating bladder and/or bowel dysfunction (and/or near other nerves/muscles at the same or different location to treat bladder and/or bowel dysfunction and/or other conditions) and/or a sensing element 3160 may be located anywhere within the torso 3120 (or other body regions) to sense physiologic information for providing patient care.

In some examples, at least a portion of the stimulation element 3150 may comprise part of an implantable component/device, such as an IPG whether full sized or sized as a microstimulator. The implantable components (e.g., IPG, other) may comprise a stimulation/control circuit, a power supply (e.g., non-rechargeable, rechargeable), communication elements, and/or other components. In some examples, the stimulation element 3150 also may comprise a stimulation electrode and/or stimulation lead connected to the implantable pulse generator.

Further details regarding a location, structure, operation and/or use of the sensing element 3160, external element(s) 3170, and/or stimulation element 3150 are described above in association with at least FIGS. 3-17.

In some examples, any one of the implantable systems or apparatuses (or a combination thereof) may be implemented as part of the example arrangement 3100 of FIG. 18 instead of, or in addition to (e.g., in complementary relation to), the stimulation element 3150, with at least some examples throughout the disclosure providing further details of such example arrangements. Moreover, at least some aspects (e.g., sensing, control, etc.) associated with an implantable system or apparatus as described in association with FIGS. 3-17 also may be implemented, in whole or part, via external element 3170 of FIG. 18.

In some examples, at least a portion of the stimulation element 3150 may comprise part of an external component/device such as, but not limited to, the external component comprising a pulse generator (e.g., stimulation/control circuitry), power supply (e.g., rechargeable, non-rechargeable), and/other components. In some examples, a portion of the stimulation element 3150 may be implantable and a portion of the stimulation element 3150 may be external to the patient.

Accordingly, as further shown in FIG. 18, the various sensing element(s) 3160 and/or stimulation element(s) 3150 implanted in the patient's body may be in wireless communication (e.g., connection 3165) with at least one external element 3170.

As further shown in FIG. 18, in some examples, the external element(s) 3170 may comprise one or more different modalities 3190 such as (but not limited to) a sensing portion 3192, stimulation portion 3194, power portion 3196, communication portion 3198, and/or other portion 3200. The different portions 3192, 3194, 3196, 3198, 3200 may be combined into a single physical structure (e.g., package, arrangement, assembly), may be implemented in multiple different physical structures, and/or with just some of the different portions 3192, 3194, 3196, 3198, 3200 combined together in a single physical structure.

Among other such details, in some examples the external sensing portion 3192 and/or implanted sensing element 3160 may comprise an example implementation of, and/or at least some of substantially the same features and attributes as, the examples further described above in association with FIGS. 3-17.

In some examples, the external stimulation portion 3194 and/or implanted stimulation element 3150 may comprise at least some of substantially the same features and attributes of at least the stimulation arrangements, as further described above in association with at least FIGS. 3-17 and/or other examples throughout the present disclosure.

In some examples, the external power portion 3196 and/or power components associated with implanted stimulation element 3150 may comprise at least some of substantially the same features and attributes of at least the stimulation arrangements, as further described in association with at least FIGS. 3-17 and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 3196 may comprise a power source by which a power component of the implanted stimulation element 3150 may be recharged.

In some examples, the wireless communication portion 3198 (e.g., connection/link at 3165) may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3200 via which other aspects of implementing medical care may be embodied in external element(s) 3170 to relate to the various implanted and/or external components described above.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1.-57. (canceled)

58. A method of treating a bladder and/or bowel dysfunction of a patient, the method comprising:
implanting a lead into the patient, the lead carrying at least one stimulation element; and
applying stimulation energy to an anatomical structure of the patient via the at least one stimulation element.

59. The method of claim 58, wherein the step of implanting includes:
loading a guidewire to a stimulation needle;
directing a tip of the stimulation needle into the patient to a location proximate a target site;
identifying a desired position of the tip relative to the target site via operation of the stimulation needle;
removing the stimulation needle from over the guidewire;
advancing an introducer over the guidewire in a direction of a distal end of guidewire such that the introducer expands contacted tissue;
removing the guidewire from the introducer; and
delivering the lead through the introducer.

60. The method of claim 59, wherein the step of loading includes inserting the guidewire into the stimulation needle.

61. The method of claim 59, wherein the target site is a branch of a pudendal nerve of the patient.

62. The method of claim 61, wherein the branch is a deep perineal branch of the pudendal nerve.

63. The method of claim 59, wherein the step of directing includes inserting the tip transperineally into the patient.

64. The method of claim 59, wherein the step of identifying includes delivering stimulation energy to the tip and monitoring a response of the patient.

65. The method of claim 58, wherein the step of implanting includes:
directing a tip of a solid body test needle into the patient to a location proximate a target site;
identifying a desired position of the tip relative to the target site via operation of the test needle;
advancing a sheath over the test needle;
removing the test needle;
advancing a lead through the sheath; and
removing the sheath.

66. The method of claim 58, wherein the step of implanting is performed via an open surgical approach.

67. The method of claim 66, wherein the open surgical approach includes:
forming a surgical cut down inferior to a gluteus maximus of the patient and into an ischiorectal fossa of the patient;
locating a target nerve via the cut down; and
implanting the lead relative to the target nerve via the cut down.

68. The method of claim 67, wherein the target nerve is a pudendal nerve of the patient.

69. The method of claim 66, wherein the open surgical approach includes:
forming a surgical cut down across a perineum of the patient;
locating a target nerve via the transperineal cut down; and
implanting the lead relative to the target nerve via the transperineal cut down;
wherein the target nerve is a pudendal nerve of the patient.

70. The method of claim 66, wherein the open surgical approach includes:
forming a surgical cut down across a vaginal wall of the patient;
locating a target nerve via the transvaginal cut down; and
implanting the lead relative to the target nerve via the transvaginal cut down.

71. The method of claim 70, wherein the target nerve is a pudendal nerve of the patient.

72. The method of claim 58, wherein the step of implanting is performed via an endoluminal approach.

73. The method of claim 72, wherein the endoluminal approach includes introducing the lead endoluminally through a transcutaneous access into a vasculature of the patient.

74. The method of claim 73, wherein the lead is introduced into a femoral vein of the patient.

75. The method of claim 73, further comprising navigating the lead through the vasculature.

76. The method of claim 75, wherein the step of navigating includes imaging the patient by at least one of fluoroscopy and ultrasonography.

77. The method of claim 75, wherein the step of navigating includes directing a distal region of the lead from a major vessel of the vasculature to a concomitant vessel adjacent to a target nerve, wherein the target nerve is one of a pudendal nerve of the patient, a splanchnic nerve of the patient, and a hypogastric nerve of the patient.
